(19) United States
(12) Patent Application Publication
(10) Pub. No.: US 2023/0165925 A1
(43) Pub. Date: Jun. 1, 2023

(54) ANTIDEPRESSANT FOOD AND METHOD FOR PREPARING THE SAME

(71) Applicant: Meyer Health Jiangsu Co., Ltd., Taizhou (CN)

(72) Inventors: Hanping SHI, Taizhou (CN); Ru JIA, Taizhou (CN)

(21) Appl. No.: 17/540,201

(22) Filed: Dec. 1, 2021

(51) Int. Cl. A61K 36/725; A61K 47/24; A61K 36/00; A61K 36/60; A61K 47/26; A61K 9/00; A61P 25/24; A23L 33/00; A23L 33/105; A23L 33/175; A23L 33/115; A23L 33/125

(52) U.S. Cl. CPC A61K 36/725; A61K 47/24; A61K 36/00; A61K 36/60; A61K 47/26; A61K 9/0056; A61P 25/24; A23L 33/40; A23L 33/105; A23L 33/175; A23L 33/115; A23L 33/125; A61K 2236/15; A61K 2236/333; A61K 2236/51; A61K 2236/39; A61K 2236/37; A61K 2236/53; A23V 2002/00

(57) ABSTRACT

An antidepressant food and a method for preparing the same are provided. The antidepressant food includes, by mass percentage, Semen ziziphi spinosae powder in a range of 28% to 36%, γ-aminobutyric acid in a range of 10% to 15%, theanine in a range of 15% to 20%, phosphatidylserine in a range of 10% to 20%, Ficus tikoua extract in a range of 8% to 14%, Huperzia serrata extract in a range of 10% to 16%, sucralose in a range of 0.5% to 1% and fruit essence in a range of 0.5% to 1.5%.

### CROSS-REFERENCE TO RELATED APPLICATIONS

### Technical Field

The present disclosure relates to the field of functional food, in particular to an antidepressant food and a method for preparing the same.

### Background

Depression is a kind of affective disorder, which is usually manifested in low mood, insomnia, lack of power, slow reaction, thought blocking, living alone behind closed doors, avoidance of social interaction, memory decline, and even suicidal tendency. And according to data shown by the World Health Organization, depression has become a kind of frequently-occurring and common disease. The latest epidemiological survey shows that the global incidence of depression has reached 4.7%, and the incidence of depression in China is also as high as 3.3%, which is increasing year by year. For depression, the common treatment methods include medication, psychotherapy, physiotherapy, among which the medication is the most extensive. At present, the commonly used antidepressants in the market are mainly a kind of tricyclic antidepressants, as the most widely used antidepressants at present, mainly including amitriptyline, doxepin and imipramine, etc., which are suitable for all kinds of depression but cause undesirable side effects such as withdrawal syndrome due to sudden withdrawal of drugs. Other heterocyclic antidepressants include mianserin, fluoxetine, paroxetine, sertraline, etc. Depression is a chronic disease, and long-term use of these antidepressants can lead to problems such as slow onset, high cost, and significant side effects such as cardiotoxicity, hepatotoxicity, and other serious allergic reactions. At the same time, these western medicines have strong side effects on human nerves, and although the patients' depression can be alleviated to some extent, but they also suffer from low spirits and mental sluggishness during the day due to the strong side effects which cause strong inhibitory effects on the nerves. According to a large number of clinical practices, about three fifths of antidepressants are of poor therapeutic effects. Compared with the western medicines, proprietary Chinese medicine has advantages of safety and less side effects, but also has disadvantages such as complex prescription, high price of some herbs, slow effect and long treatment course.

Therefore, more safe, side-effect-free, fast-acting dietetic therapy for depression has become a popular trend. As a kind of common vine, Ficus tikoua is widely distributed in Sichuan, Guangxi, Guizhou, Hunan and other places in China, and is commonly used in folk medicine. The Ficus tikoua is commonly used in treatments for chronic bronchitis, dysentery, infantile dyspepsia, rheumatism pain, unknown swelling and toxin, impetigo, scabies and so on, with curing rheumatism numbness, treating muscle and bone pain, promoting blood circulation, reducing swelling and detoxifying, having diuretic and antipyretic effects. Huperzia serrata is a perennial herbaceous plant of the family huperaceae, which is often found in wetland under shade trees, under shrubs, at roadsides or on gully rocks at an altitude of 300-2700 meters, and the whole plant is used as medicine with effects of heat clearing, detoxication, tissue regeneration, hemostasis, blood stasis dispersing and detumescence. The Huperzia serrata is used for treating traumatic injury, swelling and pain of blood stasis, internal injury and bleeding, and is used externally for treating swelling and pain of carbuncle, poisonous snake bite, burn and scald, etc.

### SUMMARY

In the present disclosure, through study, it is found that the Ficus tikoua and the Huperzia serrata can be used as optional raw materials for dietetic therapy for depression, and may be used in combination with common raw materials of antidepressant food such as Semen ziziphi spinosae, γ-aminobutyric acid, theanine and phosphatidylserine to significantly improve therapeutic effect, which is not reported in the existing technology, thereby enriching the selections of dietetic therapy for depression.

In view of the above problems, embodiments of the present disclosure provide an antidepressant food. The antidepressant food includes, by mass percentage, Semen ziziphi spinosae powder in a range of 28% to 36%, γ-aminobutyric acid in a range of 10% to 15%, theanine in a range of 15% to 20%, phosphatidylserine in a range of 10% to 20%, Ficus tikoua extract in a range of 8% to 14%, Huperzia serrata extract in a range of 10% to 16%, sucralose in a range of 0.5% to 1% and fruit essence in a range of 0.5% to 1.5%.

In one embodiment, the Ficus tikoua extract is prepared by ethanol refluxing concentration and drying of fresh Ficus tikoua.

In one embodiment, the Huperzia serrata extract is prepared by acid extraction, concentration, extraction and drying of fresh Huperzia serrata.

Embodiments of the present disclosure further provide a method for preparing an antidepressant food. The method includes filtering Semen ziziphi spinosae powder, γ-aminobutyric acid, theanine, phosphatidylserine, Ficus tikoua extract, Huperzia serrata extract, sucralose and fruit essence, respectively; blending, by mass percentage, the Semen ziziphi spinosae powder in a range of 28% to 36%, the γ-aminobutyric acid in a range of 10% to 15%, the theanine in a range of 15% to 20%, the phosphatidylserine in a range of 10% to 20%, the Ficus tikoua extract in a range of 8% to 14%, the Huperzia serrata extract in a range of 10% to 16%, the sucralose in a range of 0.5% to 1% and the fruit essence in a range of 0.5% to 1.5% to obtain the antidepressant food.

In one embodiment, the Ficus tikoua extract is prepared by chopping fresh Ficus tikoua, adding to ethanol water with mass fraction in arrange of 70% to 80% which is 5 to 10 times in weight of the fresh Ficus tikoua, fully stirring, heating to a temperature in arrange of 60° C. to 70° C. for refluxing for 1 h to 2 h, repeating three times, concentrating the refluxed liquid under reduced pressure, extracting with ethyl acetate which is 1 to 2 times in volume of the concentrated liquid, repeating three times, merging the extracted solution, concentrating under reduced pressure, and drying at a temperature in arrange of 50° C. to 60° C.

In one embodiment, the Huperzia serrata extract is prepared by grinding fresh Huperzia serrata into coarse powder, adding to hydrochloric acid aqueous solution with mass fraction in arrange of 1% to 2% which is 10 to 20 times in weight of the fresh Huperzia serrata, stirring and extracting for a time in a range of 24 h to 48 h, concentrating the extracted solution under reduced pressure, adjusting pH to a range of 8 to 9 with ammonia water, extracting for three times with chloroform, merging the extracted solution, concentrating under reduced pressure, and drying under at a temperature in arrange of 40° C. to 50° C.

In one embodiment, the filtering is performed by using a screen of 40 mesh or 60 mesh.

In one embodiment, blending, by mass percentage, the Semen ziziphi spinosae powder in a range of 28% to 36%, the γ-aminobutyric acid in a range of 10% to 15%, the theanine in a range of 15% to 20%, the phosphatidylserine in a range of 10% to 20%, the Ficus tikoua extract in a range of 8% to 14%, the Huperzia serrata extract in a range of 10% to 16%, the sucralose in a range of 0.5% to 1% and the fruit essence in a range of 0.5% to 1.5% includes adding, by mass percentage, the Semen ziziphi spinosae powder in a range of 28% to 36%, the γ-aminobutyric acid in a range of 10% to 15%, the theanine in a range of 15% to 20%, the phosphatidylserine in a range of 10% to 20%, the Ficus tikoua extract in a range of 8% to 14%, the Huperzia serrata extract in a range of 10% to 16%, the sucralose in a range of 0.5% to 1% and the fruit essence in a range of 0.5% to 1.5% into a stirrer; and stirring at a speed of 20 r/min to 40 r/min in a sealed state until no mass appears.

The advantageous effects of the present disclosure are as follows.

In the antidepressant food of the present disclosure, two new raw materials for the antidepressant food, i.e., Ficus tikoua extract and Huperzia serrata extract, are discovered on basis of common raw materials for the antidepressant food such as Semen ziziphi spinosae powder, γ-aminobutyric acid, theanine, phosphatidylserine and the like. The Ficus tikoua extract and the Huperzia serrata extract are used in in combination with the Semen ziziphi spinosae powder, the γ-aminobutyric acid, the theanine, the phosphatidylserine to obtain a new kind of food composition for depression treatment. The experimental results show that the treatment effect of the composition after adding the Ficus tikoua extract and the Huperzia serrata extract is significantly better than that without the Ficus tikoua extract and the Huperzia serrata extract. The antidepressant effect is the best when both of them are used at the same time, resulting from synergistic effect between the Ficus tikoua extract and the Huperzia serrata extract when used at the same time. In comparison with the antidepressant food commercially available at present, the food composition according to the present disclosure is of more remarkable treatment effect for depression, and further enriches the selections of dietetic therapy for depression.

Compared with western medicine and proprietary Chinese medicine in treating depression, the food provided by the present disclosure is cheaper in raw materials, less side effects, fast-acting, simple in preparation, mild in conditions, controllable, stable in process, and great in subsequent industrial production potential, and thus is promotable and applicable.

Other features and advantages of the present disclosure will be set forth in the following specification and will become apparent in part from the specification or be understood by practicing the present disclosure. The objects and other advantages of the present disclosure may be achieved and obtained by the structures indicated in the specification, the claims and the accompanying drawings.

### BRIEF DESCRIPTION OF THE DRAWINGS

In order to illustrate the technical solutions according to the embodiments of the present disclosure more clearly, the accompanying drawings for describing the embodiments are introduced briefly in the following. Apparently, the accompanying drawings in the following description are merely some embodiments of the present disclosure, and those skilled in the art may derive other drawings from the accompanying drawings without creative efforts.

FIG. 1 shows a flowchart of a method for preparing an antidepressant food according to an embodiment of the present disclosure.

### DETAILED DESCRIPTION

In order to make the objects, technical solutions and advantages of the embodiments of the present disclosure more clear, the technical solutions in the embodiments of the present disclosure will be clearly and completely described in the following with reference to the accompanying drawings in the embodiments of the present disclosure, and it will be apparent that the described embodiments are some but not all of the embodiments of the present disclosure. Other embodiments obtained by those skilled in the art without creative effort, based on the embodiments of the present disclosure, shall fall within the protection scope of the present disclosure.

The embodiments of the present disclosure provide an antidepressant food. The antidepressant food includes, by mass percentage, Semen ziziphi spinosae powder in a range of 28% to 36%, γ-aminobutyric acid in a range of 10% to 15%, theanine in a range of 15% to 20%, phosphatidylserine in a range of 10% to 20%, Ficus tikoua extract in a range of 8% to 14%, Huperzia serrata extract in a range of 10% to 16%, sucralose in a range of 0.5% to 1% and fruit essence in a range of 0.5% to 1.5%.

In one embodiment, the Ficus tikoua extract is prepared by ethanol refluxing concentration and drying of fresh Ficus tikoua.

In one embodiment, the Huperzia serrata extract is prepared by acid extraction, concentration, extraction and drying of fresh Huperzia serrata.

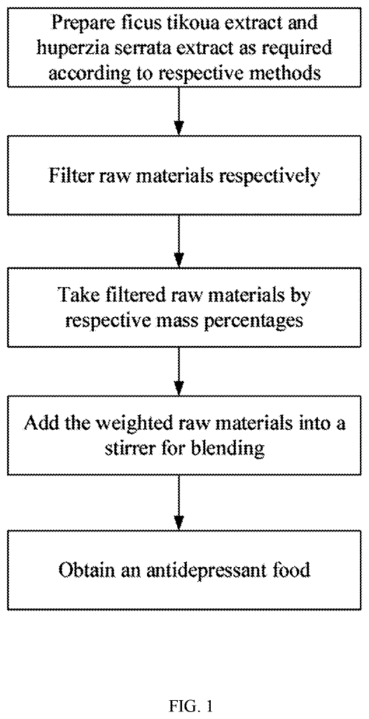

Embodiments of the present disclosure further provide a method for preparing an antidepressant food. As an example as shown in FIG. 1, the method includes steps as follows.

Semen ziziphi spinosae powder, γ-aminobutyric acid, theanine, phosphatidylserine, Ficus tikoua extract, Huperzia serrata extract, sucralose and fruit essence are filtered, respectively.

The Ficus tikoua extract is prepared by chopping fresh Ficus tikoua, adding to ethanol water with mass fraction in arrange of 70% to 80% which is 5 to 10 times in weight of the fresh Ficus tikoua, fully stirring, heating to a temperature in arrange of 60° C. to 70° C. for refluxing for 1 h to 2 h, repeating three times, concentrating the refluxed liquid under reduced pressure, extracting with ethyl acetate which is 1 to 2 times in volume of the concentrated liquid, repeating three times, merging the extracted solution, concentrating under reduced pressure, and drying at a temperature in arrange of 50° C. to 60° C.

The Huperzia serrata extract is prepared by grinding fresh Huperzia serrata into coarse powder, adding to hydrochloric acid aqueous solution with mass fraction in arrange of 1% to 2% which is 10 to 20 times in weight of the fresh Huperzia serrata, stirring and extracting for a time in a range of 24 h to 48 h, concentrating the extracted solution under reduced pressure, adjusting pH to a range of 8 to 9 with ammonia water, extracting for three times with chloroform, merging the extracted solution, concentrating under reduced pressure, and drying under at a temperature in arrange of 40° C. to 50° C.

By mass percentage, the Semen ziziphi spinosae powder in a range of 28% to 36%, the γ-aminobutyric acid in a range of 10% to 15%, the theanine in a range of 15% to 20%, the phosphatidylserine in a range of 10% to 20%, the Ficus tikoua extract in a range of 8% to 14%, the Huperzia serrata extract in a range of 10% to 16%, the sucralose in a range of 0.5% to 1% and the fruit essence in a range of 0.5% to 1.5% are blended to obtain the antidepressant food.

Specifically, the Semen ziziphi spinosae powder, the γ-aminobutyric acid, the theanine, the phosphatidylserine, the Ficus tikoua extract, the Huperzia serrata extract, the sucralose and the fruit essence are respectively filtered with a screen;

For example, the screen is of 40 mesh or 60 mesh.

With filtration, the purity of the raw materials is increased, the powder is finer, and the blending effect is better.

Specifically, the Semen ziziphi spinosae powder in a range of 28% to 36%, the γ-aminobutyric acid in a range of 10% to 15%, the theanine in a range of 15% to 20%, the phosphatidylserine in a range of 10% to 20%, the Ficus tikoua extract in a range of 8% to 14%, the Huperzia serrata extract in a range of 10% to 16%, the sucralose in a range of 0.5% to 1% and the fruit essence in a range of 0.5% to 1.5%, by mass percentage, are added into a stirrer and stirred in a sealed state until no mass appears.

For example, the stirring speed is in a range of 20 r/min to 40 r/min.

### Embodiment 1

In Embodiment 1, preparation for an antidepressant food of 100 g is taken as an example.

The antidepressant food is prepared with, by mass percentage, Semen ziziphi spinosae powder of 28%, γ-aminobutyric acid of 15%, theanine of 15%, phosphatidylserine of 20%, Ficus tikoua extract of 8%, Huperzia serrata extract of 12%, sucralose of 0.5% and fruit essence of 1.5%.

The Ficus tikoua extract is prepared by chopping fresh Ficus tikoua, adding to ethanol water with mass fraction in arrange of 70% to 80% which is 5 to 10 times in weight of the fresh Ficus tikoua, fully stirring, heating to a temperature in arrange of 60° C. to 70° C. for refluxing for 1 h to 2 h, repeating three times, concentrating the refluxed liquid under reduced pressure, extracting with ethyl acetate which is 1 to 2 times in volume of the concentrated liquid, repeating three times, merging the extracted solution, concentrating under reduced pressure, and drying at a temperature in arrange of 50° C. to 60° C.

The Huperzia serrata extract is prepared by grinding fresh Huperzia serrata into coarse powder, adding to hydrochloric acid aqueous solution with mass fraction in arrange of 1% to 2% which is 10 to 20 times in weight of the fresh Huperzia serrata, stirring and extracting for a time in a range of 24 h to 48 h, concentrating the extracted solution under reduced pressure, adjusting pH to a range of 8 to 9 with ammonia water, extracting for three times with chloroform, merging the extracted solution, concentrating under reduced pressure, and drying under at a temperature in arrange of 40° C. to 50° C.

The Semen ziziphi spinosae powder, the γ-aminobutyric acid, the theanine, the phosphatidylserine, the Ficus tikoua extract, the Huperzia serrata extract, the sucralose and the fruit essence are respectively filtered with a screen of 40 mesh.

The Semen ziziphi spinosae powder of 28 g, the γ-aminobutyric acid of 15 g, the theanine of 15 g, the phosphatidylserine of 20 g, the Ficus tikoua extract of 8 g, the Huperzia serrata extract of 12 g, the sucralose of 0.5 g and the fruit essence of 1.5 g are added into a stirrer and stirred at a speed of 20 r/min in a sealed state until no mass appears. The antidepressant food is discharged and obtained.

### Embodiment 2

In Embodiment 2, preparation for an antidepressant food of 100 g is taken as an example.

The antidepressant food is prepared with, by mass percentage, Semen ziziphi spinosae powder of 35%, γ-aminobutyric acid of 10%, theanine of 20%, phosphatidylserine of 10%, Ficus tikoua extract of 14%, Huperzia serrata extract of 10%, sucralose of 0.5% and fruit essence of 0.5%.

The preparation for the Ficus tikoua extract and the Huperzia serrata extract is the same as described in Embodiment 1.

The Semen ziziphi spinosae powder, the γ-aminobutyric acid, the theanine, the phosphatidylserine, the Ficus tikoua extract, the Huperzia serrata extract, the sucralose and the fruit essence are respectively filtered with a screen of 40 mesh.

The Semen ziziphi spinosae powder of 35 g, the γ-aminobutyric acid of 10 g, the theanine of 20 g, the phosphatidylserine of 10 g, the Ficus tikoua extract of 14 g, the Huperzia serrata extract of 10 g, the sucralose of 0.5 g and the fruit essence of 0.5 g are added into a stirrer and stirred in a sealed state until no mass appears. The antidepressant food is discharged and obtained.

### Embodiment 3

In Embodiment 3, preparation for an antidepressant food of 100 g is taken as an example.

The antidepressant food is prepared with, by mass percentage, Semen ziziphi spinosae powder of 36%, γ-aminobutyric acid of 11%, theanine of 16%, phosphatidylserine of 11%, Ficus tikoua extract of 8%, Huperzia serrata extract of 16%, sucralose of 1% and fruit essence of 1%.

The preparation for the Ficus tikoua extract and the Huperzia serrata extract is the same as described in Embodiment 1.

The Semen ziziphi spinosae powder, the γ-aminobutyric acid, the theanine, the phosphatidylserine, the Ficus tikoua extract, the Huperzia serrata extract, the sucralose and the fruit essence are respectively filtered with a screen of 60 mesh.

The Semen ziziphi spinosae powder of 36 g, the γ-aminobutyric acid of 11 g, the theanine of 16 g, the phosphatidylserine of 11 g, the Ficus tikoua extract of 8 g, the Huperzia serrata extract of 16 g, the sucralose of 1 g and the fruit essence of 1 g are added into a stirrer and stirred at a speed of 40 r/min in a sealed state until no mass appears. The antidepressant food is discharged and obtained.

### EXPERIMENTAL STUDY

### Experimental Example 1

Determination of 5-Hydroxytryptemine (5-HT) in Serum of Mice

Principle: according to monoamine hypothesis for depression, the antidepressants are effective on improvement of 5-HT nerve function, and the treatment effect of the antidepressants is determined by testing changes of 5-HT in serum of mice after the mice take antidepressants.

Samples are grouped as follows for testing.

In Group A, the antidepressant food prepared in Embodiment 1 is used.

In Group B, the antidepressant food prepared in Embodiment 1 with the Ficus tikoua extract removed is used.

In Group C, the antidepressant food prepared in Embodiment 1 with the Huperzia serrata extract removed is used.

In Group D, the Ficus tikoua extract is used.

In Group E, the Huperzia serrata extract is used.

In Group Blank, the normal saline is used.

Sixty healthy mice are randomly divided into the six groups with ten mice in each group. The mice in a respective group are fed with 1 g/kg for 28 days. Tails of mice are cut, and the blood is taken for 60 uL and kept at room temperature for 2 h to fully coagulate the blood. The coagulated blood is centrifuged at 3000 r/min at room temperature for 15 min, and then supernatant is taken and stored at a temperature of −20° C. The 5-HT in serum of mice in each group is tested according to instructions of the Enzyme Linked Immunosorbent Assay (ELISA) testing kit for 5-HT in serum of mice, and the testing results are recorded in Table 1.

TABLE 1
5-HT in Serum of Mice

| | Group | | | | | |
|---|---|---|---|---|---|---|
| | A | B | C | D | E | Blank |
| 5-HT (ng/mL) | 38.88 ± 3.64 | 22.29 ± 2.11 | 23.45 ± 2.93 | 20.33 ± 1.28 | 19.57 ± 1.46 | 16.73 ± 2.85 |

According to Table 1, compared with the normal saline in Group Blank, both the Ficus tikoua extract and the Huperzia serrata extract can increase the 5-HT in serum of mice, which indicates that the Ficus tikoua extract and the Huperzia serrata extract are effective on treatment for depression. In addition, compared with the composition of Embodiment 1, the single component of the Ficus tikoua extract and the composition of Embodiment 1 with the Ficus tikoua extract removed are less effective, and the single component of the Huperzia serrata extract and the composition of Embodiment 1 with the Huperzia serrata extract removed are also less effective, which indicates that the composition according to the present disclosure is reasonable in formula, and has synergistic effect when both the Ficus tikoua extract and the Huperzia serrata extract are used, and neither of them can be omitted.

### Experimental Example 2

Behavioral Observation in Mice

Samples are grouped as follows for testing.

In Group 1, the antidepressant food prepared in Embodiment 1 is used.

In Group 2, the antidepressant food prepared in Embodiment 2 is used.

In Group 3, the antidepressant food prepared in Embodiment 3 is used.

In Group Comparison: the antidepressant food commercially available at present is used.

In Group Blank: the normal saline is used.

Fifty healthy mice are randomly divided into the five groups according to weight balance, with ten mice in each group. The mice in a respective group are fed with 0.9 g/kg.

2.1 Forced Swimming Test in Mice

The day before the test, the mice are pre-screened by swimming. The mice are placed in a glass tank having a depth of 10 cm and a diameter of 14 cm and filled with water of a temperature of 25° C. to swim for 6 min. The mice that stop swimming for 70 to 160 seconds are selected for the formal test. The mice in each group are fed with an equal dosage for 7 days. One hour after the last feeding, the mice are subjected to swimming test. The mice are placed in the glass tank to swim for 6 min, and the immobility time when the mice stop swimming in last 4 min of the 6 min is accumulated. The results are recorded in Table 2.

TABLE 2
Immobility Time of Mice in Swimming

| Group | 1 | 2 | 3 | Comparison | Blank |
|---|---|---|---|---|---|
| Immobility | 109.3 ± 11.1 | 106.4 ± 6.2 | 116.4 ± 15.5 | 138.7 ± 6.8 | 164.4 ± 5.5 |
| Time (s) | | | | | |

According to Table 2, in the forced swimming test in mice, Groups 1 to 3 of the present disclosure can significantly shorten the immobility time compared with Group Blank. Moreover, with the same dosage, the antidepressant foods prepared according to Embodiments 1 to 3 of the present disclosure are of better treatment effect than the commercially available antidepressant food used in Group Comparison, which indicates that the antidepressant food provided by the present disclosure has stronger antidepressant activity in vivo.

2.2 Open Field Test in Mice

A cube open box (having a length of 40 cm, a width of 80 cm and a height of 80 cm) is adopted, and the side walls and bottom wall of the box are black. The bottom wall is divided into 25 equal blocks by white lines. The mice score in horizontal activity according to the number of blocks the mice pass through, and score in vertical activity according to the number of times the mice stand. Each mouse is tested once for 3 min each time. The scores of the mice in each group during the open field test are calculated and recorded in Table 3.

TABLE 3
Score of Open Field Text in Mice

| Group | 1 | 2 | 3 | Comparison | Blank |
|---|---|---|---|---|---|
| Number of | 111 ± 8 | 121 ± 9 | 106 ± 10 | 101 ± 7 | 82 ± 2 |
| Blocks the Mice | | | | | |
| Pass Through | | | | | |
| Number of | 28 ± 2 | 30 ± 3 | 27 ± 3 | 23 ± 1 | 17 ± 3 |
| Times the | | | | | |
| Mice Stand | | | | | |

According to Table 3, compared with Group Blank, the number of blocks the mice pass through and the number of times the mice stand in Groups 1 to 3 and Group Comparison are significantly increased, and the scores in in Groups 1 to 3 is higher that those in Group Comparison, which indicate that the antidepressant food provided by the present disclosure can effectively improve the exercise ability of the mice, and is more effective than the antidepressant food commercially available at present.

2.3 Mouse Tail Suspension Test

The mice are subjected to stimulation of tail suspension. During the test, the tails of the mice are clamped and suspended. The tail-suspended mice struggle to overcome the abnormal posture, but stay immobile intermittently after a period of struggle, showing despair. Each period from clamping and suspending the tails of the mice to resting is 10 min (including 6 min for adaptation and 4 min for testing). The immobility time of the mice in each group are accumulated and recorded in Table 4.

TABLE 4
Immobility Time of Mice in Tail Suspension

| Group | 1 | 2 | 3 | Comparison | Blank |
|---|---|---|---|---|---|
| Immobility | 180.4 ± 9.5 | 160.4 ± 8.4 | 185.2 ± 7.6 | 190.7 ± 7.9 | 229.3 ± 6.2 |
| Time (s) | | | | | |

According to Table 4, the immobility time during tail suspension of mice in Groups 1 to 3 and Group Comparison is significantly less than that in Group Blank, that is, the struggle activity of the mice taking the antidepressant foods prepared according to the Embodiments 1 to 3 of the present disclosure is increased, and the desire of the mice for survival is stronger, which indicate that the antidepressant food provided in the present disclosure is effective on treatment for depression.

### Experimental Example 3

Human Clinical Trial

Eighty volunteers suffering from depression are randomly divided into four groups (including Group 1, Group 2, Group 3 and Group Comparison), with twenty volunteers in each group. In Group 1, the antidepressant food prepared in Embodiment 1 of the present disclosure is used. In Group 2, the antidepressant food prepared in Embodiment 2 of the present disclosure is used. In Group 3, the antidepressant food prepared in Embodiment 3 of the present disclosure is used. In Group Comparison, the antidepressant food commercially available at present is used. Statistics of the treatment effect of each group is performed with questionnaire, and the results are recorded in Table 5.

TABLE 5
Human Clinical Trial

| Group | 1 | 2 | 3 | Comparison |
|---|---|---|---|---|
| Significantly Effective | 9 | 12 | 8 | 4 |
| Effective | 6 | 4 | 5 | 6 |
| Ineffective | 5 | 4 | 7 | 10 |

According to Table 5, the antidepressant foods provided in Embodiments 1 to 3 of the present disclosure are significantly effective on treatment for depression, and is higher in effective rate than the antidepressant food commercially available at present.

Although the present disclosure has been described in detail with reference to the foregoing embodiments, it should be appreciated for those skilled in the art should that the technical solutions described in the foregoing embodiments may be modified or some of the technical features thereof may be equivalent substituted. These modifications or substitutions do not depart from the spirit and scope of the technical solutions of the embodiments of the present disclosure.

What is claimed is:

1. An antidepressant food comprising, by mass percentage, Semen ziziphi spinosae powder in a range of 28% to 36%, γ-aminobutyric acid in a range of 10% to 15%, theanine in a range of 15% to 20%, phosphatidylserine in a range of 10% to 20%, Ficus tikoua extract in a range of 8% to 14%, Huperzia serrata extract in a range of 10% to 16%, sucralose in a range of 0.5% to 1% and fruit essence in a range of 0.5% to 1.5%.

2. The antidepressant food according to claim 1, wherein the Ficus tikoua extract is prepared by ethanol refluxing concentration and drying of fresh Ficus tikoua.

3. The antidepressant food according to claim 1, wherein the Huperzia serrata extract is prepared by acid extraction, concentration, extraction and drying of fresh Huperzia serrata.

4. A method for preparing an antidepressant food, comprising:
filtering Semen ziziphi spinosae powder, γ-aminobutyric acid, theanine, phosphatidylserine, Ficus tikoua extract, Huperzia serrata extract, sucralose and fruit essence, respectively,
blending, by mass percentage, the Semen ziziphi spinosae powder in a range of 28% to 36%, the γ-aminobutyric acid in a range of 10% to 15%, the theanine in a range of 15% to 20%, the phosphatidylserine in a range of 10% to 20%, the Ficus tikoua extract in a range of 8% to 14%, the Huperzia serrata extract in a range of 10% to 16%, the sucralose in a range of 0.5% to 1% and the fruit essence in a range of 0.5% to 1.5% to obtain the antidepressant food.

5. The method according to claim 4, wherein the Ficus tikoua extract is prepared by chopping fresh Ficus tikoua, adding to ethanol water with mass fraction in arrange of 70% to 80% which is 5 to 10 times in weight of the fresh Ficus tikoua, fully stirring, heating to a temperature in arrange of 60° C. to 70° C. for refluxing for 1 h to 2 h, repeating three times, concentrating the refluxed liquid under reduced pressure, extracting with ethyl acetate which is 1 to 2 times in volume of the concentrated liquid, repeating three times, merging the extracted solution, concentrating under reduced pressure, and drying at a temperature in arrange of 50° C. to 60° C.

6. The method according to claim 4, wherein the Huperzia serrata extract is prepared by grinding fresh Huperzia serrata into coarse powder, adding to hydrochloric acid aqueous solution with mass fraction in arrange of 1% to 2% which is 10 to 20 times in weight of the fresh Huperzia serrata, stirring and extracting for a time in a range of 24 h to 48 h, concentrating the extracted solution under reduced pressure, adjusting pH to a range of 8 to 9 with ammonia water, extracting for three times with chloroform, merging the extracted solution, concentrating under reduced pressure, and drying under at a temperature in arrange of 40° C. to 50° C.

7. The method according to claim 4, wherein the filtering is performed by using a screen of 40 mesh or 60 mesh.

8. The method according to claim 4, wherein blending, by mass percentage, the Semen ziziphi spinosae powder in a range of 28% to 36%, the γ-aminobutyric acid in a range of 10% to 15%, the theanine in a range of 15% to 20%, the phosphatidylserine in a range of 10% to 20%, the Ficus tikoua extract in a range of 8% to 14%, the Huperzia serrata extract in a range of 10% to 16%, the sucralose in a range of 0.5% to 1% and the fruit essence in a range of 0.5% to 1.5% comprises:
adding, by mass percentage, the Semen ziziphi spinosae powder in a range of 28% to 36%, the γ-aminobutyric acid in a range of 10% to 15%, the theanine in a range of 15% to 20%, the phosphatidylserine in a range of 10% to 20%, the Ficus tikoua extract in a range of 8% to 14%, the Huperzia serrata extract in a range of 10% to 16%, the sucralose in a range of 0.5% to 1% and the fruit essence in a range of 0.5% to 1.5% into a stirrer, and
stirring at a speed of 20 r/min to 40 r/min in a sealed state until no mass appears.
